(19) United States
(12) Patent Application Publication
(10) Pub. No.: US 2014/0079648 A1
(43) Pub. Date: Mar. 20, 2014

(54) ANTIMICROBIAL ALCOHOLIC FOAMABLE COMPOSITIONS

(71) Applicant: GOJO INDUSTRIES, INC., Akron, OH (US)

(72) Inventor: Mitchell Jared Cohen, Copley, OH (US)

(73) Assignee: GOJO INDUSTRIES, INC., Akron, OH (US)

(21) Appl. No.: 13/828,174

(22) Filed: Mar. 14, 2013

Related U.S. Application Data
(60) Provisional application No. 61/701,073, filed on Sep. 14, 2012.

(51) Int. Cl. A61K 9/12

(52) U.S. Cl. CPC A61K 9/122
USPC 424/45

(57) ABSTRACT

An antimicrobial alcoholic foamable composition comprising an alcohol, a waxy component, water, and a dissolvable gas, where the composition is dispensed and foamed without requiring the use of a porous filter, meshed screen, or hazardous propellant.

### TECHNICAL FIELD

Embodiments of the present invention provide antimicrobial alcoholic foamable compositions, methods for dispensing antimicrobial alcoholic foam compositions, and methods for producing antimicrobial alcoholic foam compositions.

### BACKGROUND OF THE INVENTION

There is a need in healthcare settings for efficacious broad-spectrum antimicrobial disinfection products. Foam cleaning products are popular, in part because they are easier to spread on surfaces. Consumers seem to prefer the luxury of foamed soap and disinfecting products. Less foam is needed to produce the same cleaning power as liquids or gels, due at least partly to the higher surface area of the foam. Properly formulated foam products do not produce the drip and splash that is experienced with traditional gelled or liquid products, because the formulation is not dispensed in a liquid state. This prevents damage to the floors and walls of facilities where the product dispensers are used. Manufacturing of foam products may be easier than gelled products, which often incorporate powdered thickeners that are difficult to handle.

Typically, alcohol foam products have been either aerosol or non-aerosol. Non-aerosol foam products require dispensers that are adapted to mix a gas with a liquid in order to produce a foam. U.S. Pat. No. 6,660,282 describes some of the drawbacks of non-aerosol foam products. The '282 patent states that certain types of non-aerosol dispensers operate with porous filters or meshed screens, and therefore are not tolerant to particulate components or to even modestly viscous compositions. The '282 patent also states that non-aerosol generated foams often lack a dense luxurious character and often do not provide a cushioned afterfeel.

Aerosol foam products require a pressurized system and a propellant. Commonly used propellants include hydrocarbons.

Thus, there is a need in the art for an antimicrobial foam product that can be dispensed and foamed without the use of a porous filter or mesh screen and that can be dispensed without the use of a hydrocarbon propellant.

### SUMMARY OF THE INVENTION

Embodiments of the present invention provide a foamable antimicrobial alcoholic compositions wherein a dissovable, non-hydrocarbon gas is dissolved in a hydroalcoholic liquid composition under slight pressure, wherein upon release of the pressure, an antimicrobial alcoholic foam is formed.

Embodiments of the present invention provide a foamable antimicrobial alcoholic foamable composition comprising a liquid phase that includes alcohol, a waxy component, water, and a non-hydrocarbon gas dissolved in the liquid phase under slight pressure.

In one or more embodiments, the waxy component is selected from the group consisting of emulsifying wax, beeswax, carnauba wax, candelilla wax, ouricury wax, Japan wax, cork fiber wax, sugarcane wax, paraffin waxes, lignite waxes, microcrystalline waxes, lanolin wax, ozokerite, polyethylene wax, silicone waxes, plant waxes, C₈₋₂₄ fatty alcohols, C₈₋₂₄ fatty acids, glycerides that are solid at room temperature, blends of a C₈₋₂₄ fatty alcohol with an ethoxylated C₈₋₂₄ fatty alcohol, blends of a C₈₋₂₄ fatty alcohol and a non-ionic surfactant, and mixtures thereof.

In any of the above embodiments, or as further described herein, the composition further comprises an emulsifier selected from the group consisting of polysorbate, polaxamer, polyethoxylated siloxane polymer, and mixtures thereof.

In any of the above embodiments, or as further described herein, the dissolvable gas is selected from the group consisting of carbon dioxide, nitrous oxide, and mixtures thereof.

In any of the above embodiments, or as further described herein, the composition is a saturated solution with respect to the dissolvable gas.

In any of the above embodiments, or as further described herein, the waxy component is present in an amount of at least 0.05 wt. %, based upon the total weight of the liquid phase.

In any of the above embodiments, or as further described herein, the waxy component is present in an amount of at least about 0.1 wt. %, based upon the total weight of the liquid phase.

In any of the above embodiments, or as further described herein, the composition comprises at least about 40 wt. % alcohol, based upon the total weight of the liquid phase.

In any of the above embodiments, or as further described herein, the composition comprises at least about 0.01 wt. % water, based upon the total weight of the liquid phase.

In any of the above embodiments, or as further described herein, the composition comprises from about 0.1 to about 10 grams dissolvable gas, per 100 grams of the liquid phase.

In any of the above embodiments, or as further described herein, the foam expands upon the release of pressure to a final volume that is from about 1.5 or more to about 5 times the liquid volume of the pressurized composition.

In any of the above embodiments, or as further described herein, the foam is stable for at least 1 minute at room temperature and pressure.

Embodiments of the present invention provide a method of preparing an antimicrobial alcoholic foamable composition, the method comprising the steps of: providing a dispenser capable of being pressurized; filling said dispenser with a liquid composition comprising an alcohol, a waxy component, and water; adding a dissolvable gas; thereby pressurizing said dispenser to a pressure greater than atmospheric pressure at standard temperature; whereupon dispensing an aliquot of said composition a foam is formed.

### BRIEF DESCRIPTION OF THE DRAWINGS

FIG. 1 is a photograph of the foam produced according to Sample 1.

FIG. 2 is a photograph of the foam produced according to Sample 2.

FIG. 3 is a photograph of the foam produced according to Sample 3.

### DETAILED DESCRIPTION OF ILLUSTRATIVE EMBODIMENTS

In one or more embodiments, the present method provides an antimicrobial alcoholic foamable composition. In addition to being effective as a hand sanitizer, the antimicrobial composition of the present invention may be employed on a wide variety of surfaces or substrates, including skin, porous, and non-porous surfaces.

The antimicrobial alcoholic foamable composition includes a liquid phase and a gas dissolved in the liquid under slight pressure. In one or more embodiments, the liquid phase comprises an alcohol, a waxy component, and water.

In one or more embodiments, the alcohol is a lower alcohol, i.e. an alcohol containing 1 to 6 carbon atoms. Typically, these alcohols have antimicrobial properties. Examples of lower alcohols include, but are not limited to, methanol, ethanol, propanol, butanol, pentanol, hexanol, and isomers and mixtures thereof. In one or more embodiments, the alcohol comprises ethanol, propanol, or butanol, or isomers or mixtures thereof. In particular embodiments, the alcohol comprises ethanol.

In one or more embodiments, the liquid phase comprises at least 40 percent by weight (wt. %) C₁₋₆ alcohol, based upon the total weight of the liquid phase, in other embodiments, at least 50 wt. %, in other embodiments, at least 60 wt. %, in other embodiments, at least 70 wt. %, in yet other embodiments, at least 80 wt. %, and in other embodiments, at least 90 wt. % alcohol, based upon the total weight of liquid phase. More or less alcohol may be required in certain instances, depending particularly on other ingredients and/or the amounts thereof employed in the liquid phase. In one or more embodiments, the liquid phase comprises from about 50 to about 99 wt. % of C₁₋₆ alcohol, in other embodiments, from about 60 to about 98 wt. %, in yet other embodiments, from about 70 to about 97 wt. %, and in still other embodiments, from about 80 to about 96 wt. % of C₁₋₆ alcohol, based upon the total weight of the liquid phase.

In one or more embodiments, the composition comprises a waxy component. The waxy component may be an emulsifying wax or a mixture or blend of a wax and an emulsifier.

In one or more embodiments, the waxy component may be a lipophilic fatty compound that is solid at room temperature (25° C.) and atmospheric pressure (760 mmHg, i.e. 101 kPa). Typically, the waxy component has a melting point greater than 30° C., in other embodiments, greater than 35° C., in other embodiments, greater than 40° C. In one or more embodiments, the waxy component is characterized by an anisotropic crystal organization in its solid state. Many waxes are capable of undergoing a reversible solid/liquid change of state. Generally, the waxy component is insoluble in water. In one or more embodiments, the waxy component may be selected from natural waxes, synthetic waxes, and emulsifying waxes. Examples of waxes include polyethylene waxes derived from ethylene (co)polymerisation, waxes obtained by Fischer-Trops synthesis, fatty alcohols, fatty acid esters, glycerides, silicone waxes, and mixtures thereof

Examples of useful waxes also include emulsifying waxes. In general, while waxes per se are substantially hydrophobic, emulsifying waxes include waxes that have been chemically treated and/or blended with additional ingredients in order to enhance the ability of the wax to act as an emulsifier. Emulsifying waxes that comply with the standards of the National Formulary are sometimes referred to as emulsifying wax NF. Examples of emulsifying waxes include blends of one or more fatty alcohol and one or more emulsifying agent.

In one or more embodiments, the waxy component is selected from the group consisting of beeswax, carnauba wax, candelilla wax, ouricury wax, Japan wax, cork fiber wax, sugarcane wax, paraffin waxes, lignite waxes, microcrystalline waxes, lanolin wax, ozokerite, polyethylene wax, silicone waxes, plant waxes, C₈₋₂₄ fatty alcohols, C₈₋₂₄ fatty acids, glycerides that are solid at room temperature, blends of a C₈₋₂₄ fatty alcohol with an ethoxylated C₈₋₂₄ fatty alcohol, blends of a C₈₋₂₄ fatty alcohol and a non-ionic surfactant, and mixtures thereof In one or more embodiments, the waxy component includes capryl alcohol, pelargonic alcohol, decyl alcohol, undecyl alcohol, lauryl alcohol, tridecyl alcohol, myristyl alcohol, pentadecyl alcohol, cetyl alcohol, palmitoleyl alcohol, heptadecyl alcohol, stearyl alcohol, isostearyl alcohol, oleyl alcohol, linoleyl alcohol, linolenyl alcohol, ricinoleyl alcohol, nonadecyl alcohol, arachidyl alcohol, heneicosyl alcohol, behenyl alcohol, erucyl alcohol, lignoceryl alcohol, cetearyl alcohol, or a combination thereof. In one or more embodiments, the waxy component is a blend that includes cetearyl alcohol and ceteareth 20. In one or more embodiments, the waxy component is a blend that includes cetearyl alcohol and stearyl alcohol. In one or more embodiments, the waxy component is a blend that includes cetearyl alcohol, polysorbate 60, PEG-150 stearate, and steareth-20.

In one or more embodiments, the emulsifying wax is a mixture of cetearyl alcohol and ceteareth 20. In one or more embodiments, the emulsifying wax is a mixture of cetearyl alcohol and stearyl alcohol. In one or more embodiments, the emulsifying wax is a mixture of cetearyl alcohol, polysorbate 60, PEG-150 stearate, and steareth-20.

The amount of waxy component is not particularly limited, but may be selected based upon such factors as aesthetic preferences and solubility parameters. In one or more embodiments, the amount of waxy component may be expressed relative to the total weight of liquid phase. In these or other embodiments, the amount of waxy component is at least about 0.05 wt. %, in other embodiments at least about 0.1 wt. %, in other embodiments at least about 0.2 wt. %, in other embodiments at least about 0.5 wt. %, and in other embodiments at least about 0.8 wt. %, based upon the total weight of the liquid phase. In these or other embodiments, the amount of waxy component is from about 0.2 to about 10 wt. %, in other embodiments from about 0.5 to about 8 wt. %, in other embodiments from about 0.8 to about 6 wt. %, based upon the total weight of the liquid phase.

In one or more embodiments, the liquid phase may further include an emulsifying agent.

Examples of emulsifying agents include non-ionic surfactants, ethoxylated fatty alcohols and ethoxylated fatty alcohol esters. In one embodiment, the emulsifying agent is a polysorbate, polaxamer, polyethoxylated siloxane polymer, or a mixture thereof. In one embodiment, the emulsifying agent is sorbitan oleate trideceth-2, polysorbate 60, PEG-150 stearate, steareth-20, ceteareth-20, PEG/PPG-20/6 dimethicone, or a mixture thereof.

In one or more embodiments, the emulsifier is characterized by an HLB of from 1 to 20.

In one or more embodiments, where the foamable alcoholic composition includes an emulsifying agent, the emulsifying agent may be present in an amount of from about 0 to about 10 wt. %, in other embodiments from about 0.1 to about 5 wt. %, in other embodiments from about 0.5 to about 2 wt. %, based upon the total weight of the liquid phase.

In one or more embodiments, the balance of the liquid phase is water. In other embodiments, the liquid phase may include one or more optional ingredients, with the proviso that there is no deleterious effect on the foaming or antimicrobial efficacy of the antimicrobial foam.

In one or more embodiments, the foamable alcoholic composition comprises at least about 0.01 wt. % water, based upon the total weight of the liquid phase. In one or more embodiments, the composition comprises at least about 1 wt. %, in other embodiments, at least about 10 wt. %, in other embodiments, at least about 20 wt. %, in other embodiments, at least about 40 wt. %, and in other embodiments, at least about 50 wt. %, based upon the total weight of liquid phase. In one or more embodiments, the foamable alcoholic composition comprises from about 0.01 to about 55 wt. %, in other embodiments, from about 1 to about 50 wt. %, and in yet other embodiments, from about 10 wt. % to about 35 wt. % of water, based upon the total weight of the liquid phase. In one or more embodiments, the balance of the liquid phase or composition is water.

In one or more embodiments, the gas component comprises a dissolvable gas. It should be appreciated that the dissolvable gas is added to the liquid phase by methods that are known in the art. In one or more embodiments, the dissolvable gas is not produced by a chemical reaction within the composition nor is the dissolvable gas created through a fermentation process.

In one or more embodiments, the gas component comprises a dissolvable gas where the dissolvable gas is not a hydrocarbon, chlorofluorocarbon, fluorocarbon, or hydrofluorocarbon.

Suitable dissolvable gases include those that are capable of dissolving into the antimicrobial alcoholic foamable composition under slight pressure, and standard temperature. In one or more embodiments, the dissolvable gas is at least partially soluble in water at standard temperature and pressure. In one or more embodiments, the dissolvable gas is at least partially soluble in the antimicrobial alcoholic foamable composition under slight pressure. In one or more embodiments, the dissolvable gas is at least partially soluble in the antimicrobial alcoholic foamable composition at a pressure of about 40 pounds per square inch (psig), in other embodiments, the dissolvable gas is at least partially soluble in the antimicrobial alcoholic foamable composition at a pressure of about 30 psig, in other embodiments, the dissolvable gas is at least partially soluble in the antimicrobial alcoholic foamable composition at a pressure of about 20 psig, in other embodiments, the dissolvable gas is at least partially soluble in the antimicrobial alcoholic foamable composition at a pressure of about 10 psig, at standard temperature.

In one or more embodiments, the dissolvable gas is inert with respect to the other components of the composition. Examples of suitable dissolvable gases include carbon dioxide, nitric oxide, nitrogen, and compressed air.

It will be understood that some dissolvable gases have limited solubility in the liquid phase and in antimicrobial alcoholic compositions, and that the maximum amount of dissolvable gas may be defined by the solubility limits at a certain temperature and pressure.

Generally, the foamable alcoholic composition includes an effective amount of one or more dissolvable non-hydrocarbon gases. In one or more embodiments, the effective amount of dissolvable gas may be defined as the minimum amount of dissolvable gas that is effective to produce a foam when the composition is dispensed at standard temperature and pressure.

In one or more embodiments, the effective amount of dissolvable gas may be expressed relative to the amount of the liquid phase. In one or more embodiments, the effective amount of dissolvable gas is at least about 0.1 gram (g), in other embodiments, at least about 0.5 g, and in other embodiments, at least about 1 g, per 100 g of the total weight of the liquid phase of the antimicrobial alcoholic foamable composition. In one or more embodiments, the amount of dissolvable gas is from about 0.1 to about 10 g, per 100 g of the liquid phase. In one or more embodiments, the liquid phase is saturated with respect to the dissolvable gas, at standard temperature and at the pressure within the dispenser.

As described hereinabove, the antimicrobial alcoholic foamable composition of this invention includes a number of ingredients that combine to produce an antimicrobial alcoholic foam. The antimicrobial alcoholic foamable composition may further comprise a wide range of optional ingredients, with the proviso that they do not deleteriously affect the foaming or sanitizing efficacy of the compositions. The CTFA International Cosmetic Ingredient Dictionary and Handbook, Eleventh Edition 2005, and the 2004 CTFA International Buyer's Guide, both of which are incorporated by reference herein in their entirety, describe a wide variety of non-limiting cosmetic and pharmaceutical ingredients commonly used in the skin care industry, that are suitable for use in the compositions of the present invention. Non-limiting examples of functional classes of ingredients are described at page 537 of this reference. Examples of these functional classes include: abrasives, anti-acne agents, anti-caking agents, antioxidants, binders, biological additives, bulking agents, chelating agents, chemical additives; colorants, conditioners, cosmetic astringents, cosmetic biocides, denaturants, drug astringents, emulsifiers, external analgesics, film formers, fragrance components, humectants, moisturizers, opacifying agents, plasticizers, preservatives (sometimes referred to as antimicrobials), propellants, reducing agents, skin bleaching agents, skin-conditioning agents (emollient, miscellaneous, and occlusive), skin protectants, solvents, surfactants, foamableboosters, hydrotropes, solubilizing agents, suspending agents (nonsurfactant), sunscreen agents, ultraviolet light absorbers, detackifiers, and viscosity increasing agents (aqueous and nonaqueous). Examples of other functional classes of materials useful herein that are well known to one of ordinary skill in the art include sequestrants, keratolytics, topical active ingredients, and the like.

In certain embodiments, the antimicrobial composition comprises one or more humectants. Examples of humectants include propylene glycol, dipropyleneglycol, hexylene glycol, 1,4-dihydroxyhexane, 1,2,6-hexanetriol, sorbitol, butylene glycol, alkanediols, such as methyl propane diol and 1,2-octanediol, dipropylene glycol, triethylene glycol, glycerin (glycerol), polyethylene glycols, ethoxydiglycol, polyethylene sorbitol, and combinations thereof. Other humectants include glycolic acid, glycolate salts, lactate salts, lactic acid, sodium pyrrolidone carboxylic acid, hyaluronic acid, chitin, and the like.

In one embodiment, the antimicrobial alcoholic foamable composition includes one or more thickeners and optionally one or more stabilizers. Examples of thickeners and stabilizers include hydroxyethyl cellulose, hydroxypropyl cellulose, methyl cellulose, carboxymethyl cellulose, and ammonium acryloyldimethyltaurateNP copolymer. In one embodiment, where the thickener or stabilizer is starch-based, the thickener or stabilizer may be present in an amount of up to about 10 wt. %, in another embodiment in an amount of from about 0.1 to about 5 wt. %, in yet another embodiment from about 0.2 to about 1 wt. %, based upon the total weight of the liquid phase. In other embodiments, where the thickener or stabilizer is a synthetic polymer, the thickener or stabilizer may be present in an amount of up to about 15 wt. %, in another embodiment in an amount of from about 0.1 to about 10 wt. %, in yet another embodiment from about 1 to about 2 wt. %, based upon the total weight of the liquid phase.

In one or more embodiments, the antimicrobial alcoholic foamable composition has a viscosity of less than about 1000 centipoise (cps), in other embodiments, less than about 800 cps, in other embodiments, less than about 500 cps. The viscosity may be measured by a Brookfield RV Viscometer using RV and/or LV Spindles at 22° C.+/−3° C.

In one or more embodiments, the antimicrobial composition includes one or more solubilizers. Examples of solubilizers include PEG-40 hydrogenated castor oil, polysorbate-80, PEG-80 sorbitan laurate, ceteareth-20, oleth-20, PEG-4, and propylene glycol. The amount of solubilizer is not particularly limited, so long as it does not deleteriously affect the foaming or antimicrobial efficacy of the composition.

In one or more embodiments, the liquid phase and/or antimicrobial alcoholic foamable composition includes one or more antiviral agents or antiviral enhancers. Examples of antiviral enhancers include cationic oligomers and polymers, chaotropic agents, and copper and zinc compounds. Antiviral enhancers are further described in U.S. Pat. No. 8,119,115 and co-pending U.S. Patent Application Publications 2007/0185216, and 2009/0018213, all of which are hereby incorporated by reference.

In certain embodiments, the antimicrobial foamable composition does not contain any auxiliary antimicrobial ingredients, wherein the term auxiliary antimicrobial agents refers to antimicrobial agents other than C₁₋₆ alcohols. In one embodiment, the amount of auxiliary antimicrobial agent (including preservatives) is less than about 0.1 wt. %, in another embodiment, less than about 0.05 wt. %, based upon the total weight of the antimicrobial composition. In another embodiment, the antimicrobial composition is devoid of auxiliary antimicrobial agents.

It is envisioned that, in other embodiments, auxiliary antimicrobial agents could be included, with the proviso that the antimicrobial ingredient does not deleteriously affect the sanitizing properties of the composition. Auxiliary antimicrobial agents and antimicrobial enhancers are further described in co-pending U.S. Patent Application Publication 2012/0129950 and International Patent Application Publication WO 2011/119517, both of which are incorporated herein by reference.

In one or more embodiments, the foamable composition may be prepared by simply mixing the ingredients together. In one or more embodiments, where the waxy component is obtained as a solid, the waxy component may be heated above its melting temperature prior to adding the waxy component to the other ingredients. In one or more embodiments, the waxy component is heated to at least 45 degrees Celsius (° C.), prior to addition.

In one or more embodiments, the waxy component and any excipients that are added to the composition are added after the alcohol has been added to the composition.

The antimicrobial alcoholic foamable composition may be stored within a dispenser that has been pressurized. In one or more embodiments, when the dispenser is actuated and an aliquot of the composition is dispensed, the pressure gradient between the slightly elevated pressure within the dispenser and the atmospheric pressure outside the dispenser causes the dissolved gas to be released from the liquid. Advantageously, it is not necessary to mix air with the liquid aliquots. It is believed that the movement of the dissolved gas out of solution causes the antimicrobial alcoholic composition to form a foam.

In one or more embodiments, the pressure within the filled dispenser at standard temperature is greater than atmospheric pressure, in other embodiments, greater than about 10 psig, in other embodiments, greater than about 20 psi. In one or more embodiments, the pressure within the filled dispenser is less than about 50 psi, in other embodiments, less than about 40 psi, in other embodiments, less than about 35 psi. In one or more embodiments, the pressure within the dispenser containing the antimicrobial alcoholic foamable composition is from about 10 psig to about 50 psig, in other embodiments, from about 20 to about 40 psig, in other embodiments, from about 25 to about 35 psi.

In particular embodiments, the aliquots are dispensed through orifices that are configured to enable the releases an aliquot of a particular volume. It will be understood that the dispenser may take a variety of forms, and may include a variety of components and configurations in order to dispense the desired aliquots of the foam composition. Advantageously, it is not necessary to use a mesh or screen.

Methods for using pressurized containers for mixing substances with carbon dioxide and dispensing are described in U.S. Pat. No. 7,077,297, which is incorporated by reference herein. In one or more embodiments, the dispenser is a soda siphon, such as those commercially available from iSi North America under the tradename Twist 'N Sparkle™.

In one or more embodiments, the composition may be dispensed in a desired amount. In one or more embodiments, the size of the aliquot dispensed may be selected to provide an advantageous amount for a personal use.

In one or more embodiments, the amount of the aliquot may be selected to provide about 1 gram (g) or more of the antimicrobial alcoholic foamable composition, in other embodiments about 2 g or more, in other embodiments about 3 g or more, in other embodiments about 4 g or more for each engagement of the dispenser.

In one or more embodiments, the methods of the present invention produce stable foams, where foam stability is measured as the amount of time before the foam breaks down into a liquid, when dispensed onto a surface at standard temperature and pressure and observed over a period of time. In particular embodiments, the foam stability is about 1 minute or more, in other embodiments about 5 minutes or more, in other embodiments about 10 minutes or more, in other embodiments about 15 minutes or more, in other embodiments about 20 minutes or more.

In one or more embodiments, the foam is creamy and white, although it will be understood that pigments or dyes could be added to the composition to produce a colored foam. The bubbles are small and substantially uniform. The volume expansion of the foam, when compared to the volume of the antimicrobial alcoholic composition, is significant, and in one or more embodiments is about 150% or more, in other embodiments about 200% or more, in other embodiments about 300% or more. In one or more embodiments, the foam expands upon the being released from the pressurized dispenser to a final volume that is about 1.5 to about 5 times the liquid volume of the composition.

Methods of the present invention can advantageously be employed to produce foams that contain high amounts of lower alcohols, such as ethanol, and which thus have significant antimicrobial efficacy.

In order to demonstrate the practice of the present invention, the following examples have been prepared and tested. The examples should not, however, be viewed as limiting the scope of the invention. The claims will serve to define the invention.

### EXAMPLES

Example 1 was prepared by combining 65.5 grams (g) ethanol, 32.75 g water, and 1.75 g PEG-10 dimethicone in the amounts shown in Table 1, and mixing until a homogeneous solution was achieved. The composition was placed into an Airspray® non-aerosol foam pump and an aliquot was dispensed. A photograph was taken and is shown in FIG. 3.

Example 2 was a commercially available aerosol foam. According to the label, Example 2 contains: 62.5% v/v ethanol, H₂O, hydrofluorocarbon 152A, isobutene, emulsifying wax NF, propane, cetyl lactate, steareth-2, sodium benzoate, sodium sesquicarbonate, and fragrance. The amounts of each ingredient other than the ethanol are unknown. Example 2 was contained in a dispenser that was adapted to maintain the composition under pressure, and to dispense an aliquot of the composition when the dispenser was activated. An aliquot of the composition was dispensed, and the results was a creamy, white homogeneous foam. A photograph of the foam was taken, and is shown in FIG. 2.

Example 3 was prepared by combining 458.5 g ethanol, 14.0 g emulsifying wax, and 227.5 g water, and mixed until homogeneous. The emulsifying wax employed for Example 3 was Polawax A-31, available from Croda Inc. Product information from the supplier indicates that Polawax A-31 is an emulsifying wax that contains a proprietary blend of a waxy component and an emulsifier. All solutions were clear, homogeneous liquids, free from particulate or sediment. The total volume was approximately 1 liter. Approximately 700 g of the liquid was placed into a Twist 'N Sparkle™ soda siphon. Carbon dioxide gas (8 g) was injected into the soda siphon. When an aliquot was dispensed from the container, the result was a creamy, white homogeneous foam, with good volume, that maintained foam volume and did not collapse into a liquid for at least 5 minutes. A photograph was taken, and is shown in FIG. 3. The volume of the foam was more than twice the volume of the original composition. The foam retained its shape and did not collapse to a liquid after at least 15 minutes. That is, the foam produced by Examples 3 was essentially unchanged after 15 minutes of observation.

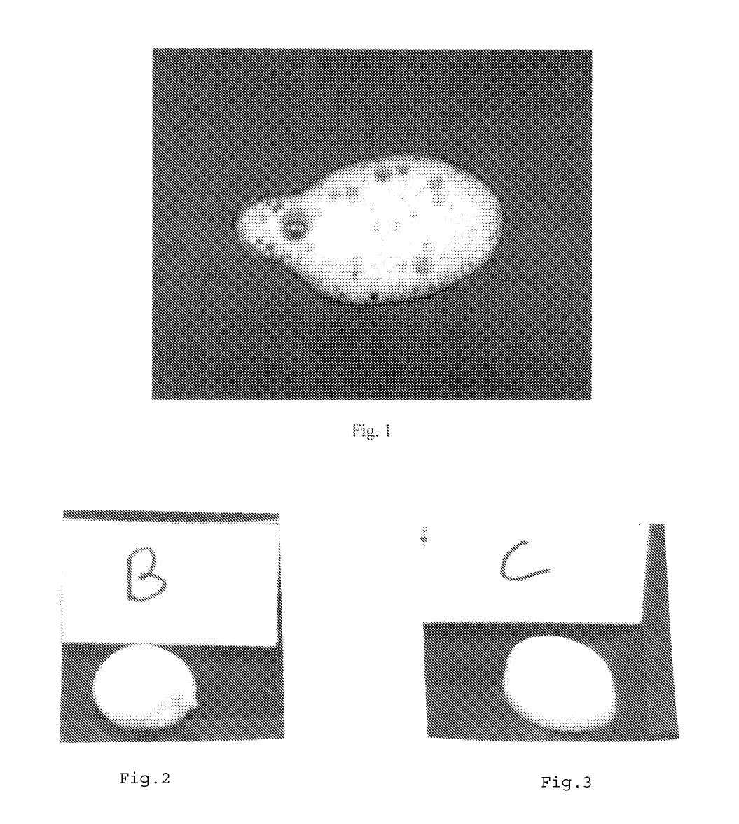

FIG. 1 is a photograph of the foam that was produced when Example 1 was dispensed using an Airspray® dispenser, and the photograph was taken immediately after dispensing. FIG. 2 shows an image of the foam that was produced when Example 2 was dispensed using an aerosol dispenser, and the photograph was taken immediately after dispensing. FIG. 3 shows an image of the foam that was produced when Example 3 was dispensed using a Twist 'N Sparkle™ dispenser, and the photograph was taken immediately after dispensing.

Advantageously, the methods of the present invention are useful to produce high quality stable alcoholic foams, without the need for mixing the product with air, passing the liquid through a mesh or screen, or hazardous propellants.

Various modifications and alterations that do not depart from the scope and spirit of this invention will become apparent to those skilled in the art. This invention is not to be duly limited to the illustrative embodiments set forth herein.

What is claimed is:

1. An antimicrobial alcoholic foamable composition, wherein the composition comprises:
(a) an alcohol,
(b) a waxy component,
(c) water, and
(d) a dissolvable gas.

2. The composition of claim 1, wherein the waxy component is an emulsifying wax.

3. The composition of claims 1, wherein the waxy component is a mixture of a wax and an emulsifier.

4. The composition of claim 1, wherein said wax is selected from the group consisting of beeswax, carnauba wax, candelilla wax, ouricury wax, Japan wax, cork fiber wax, sugarcane wax, paraffin waxes, lignite waxes, microcrystalline waxes, lanolin wax, ozokerite, polyethylene wax, silicone waxes, plant waxes, C₈₋₂₄ fatty alcohols, C₈₋₂₄ fatty acids, glycerides that are solid at room temperature, blends of a C₈₋₂₄ fatty alcohol with an ethoxylated C₈₋₂₄ fatty alcohol, blends of a C₈₋₂₄ fatty alcohol and a non-ionic surfactant, and mixtures thereof.

5. The composition of claim 4, wherein the composition further comprises an emulsifier selected from the group consisting of polysorbate, polaxamer, polyethoxylated siloxane polymer, or a mixture thereof.

6. The composition of claims 5, wherein the emulsifier is a poloxamer.

7. The composition of claim 1, wherein the dissolvable gas is selected from the group consisting of carbon dioxide, nitrous oxide, and mixtures thereof.

8. The composition of claim 1, wherein the composition is a saturated solution with respect to the dissolvable gas.

9. The composition of claim 1, wherein the composition is contained in a dispenser under slighty elevated pressure.

10. The composition of claim 1, wherein the waxy component is present in an amount of at least about 0.2 wt. %, based upon the total weight of the composition.

11. The composition of claim 1, wherein the waxy component is present in an amount of at least about 0.1 wt. %, based upon the total weight of the composition.

12. The composition of claim 1, wherein the composition comprises at least about 40 wt. % alcohol.

13. The composition of claim 1, wherein the composition comprises at least about 0.01 wt. % water.

14. The composition of claim 1, wherein the composition comprises from about 0.1 to about 10 g dissolvable gas, per 100 g combined of alcohol, water, and waxy component.

15. A method of preparing an antimicrobial alcoholic foamable composition, the method comprising the steps of:
providing a dispenser;
filling said dispenser with a liquid composition comprising
an alcohol,
a waxy component,
water, and
adding a dissolvable gas; thereby pressurizing said composition to a pressure slightly above atmospheric pressure; wherein said foamable composition forms a foam upon being dispensed from said dispenser.

16. The method of claim 15, wherein the composition is pressurized to at least about 10 psi within the dispenser.

17. The method of claim 15, wherein the waxy component is an emulsifying wax.

18. The method of claim 15, wherein the waxy component is a mixture of a wax and an emulsifier.

19. The method of claim 15, wherein the foam expands to a final volume that is about 1.5 to about 5 times the volume of the liquid composition.

20. The method of claim 15, wherein the foam is stable for at least 1 minute at room temperature and pressure.
